(19) United States
(12) Patent Application Publication
(10) Pub. No.: US 2003/0032177 A1
(43) Pub. Date: Feb. 13, 2003

(54) Cell maintenance and proliferation

(76) Inventors: Stephen Dalton, Westbourne Park (AU); Peter John Cartwright, Unley (AU)

Correspondence Address: Sutherland Asbill & Brennan, LLP, 999 Peachtree St. N.E., Atlanta, GA, 30309, US

(21) Appl. No.: 10/277,011

(22) Filed: Oct. 21, 2002

Related U.S. Application Data
(63) Continuation-in-part of application No. 10/089,130.
Application No. 10/089,130 is a 371 of international application No. PCT/AU00/01184, filed on Sep. 22, 2000.

(30) Foreign Application Priority Data
Sep. 24, 1999 (AU) PQ 3073

(51) Int. Cl. C12N 5/06; C12N 5/08

(52) U.S. Cl. 435/354; 435/366

(57) ABSTRACT

A method of regulating the differentiation potential of a pluripotent or pluripotent-related cell which method Includes manipulating the expression and/or activity of a cell differentiation regulatory molecule in a pluripotent or pluripotent related cell.

The present invention relates to methods for using molecules relating to the control of cell proliferation, to improve technology relating to pluripotent and multipotent cells. More particularly, the present invention relates to methods for enhancing the maintenance and proliferation of pluripotent cells.

Also within the scope of the present invention are cells, embryos and animals produced using the methods referred to above. In addition, uses of the cells, embryos and animals derived by these methods are within the scope of this invention.

In this patent application the term “pluripotent” refers to cells that can contribute substantially to all tissues of the developing embryo. “Multipotent” refers to partially differentiated cells that are able to differentiate further into more than one terminally differentiated cell type.

“Maintenance of pluripotent cells” is to be understood as the maintenance of such cells in vitro in an undifferentiated state. It may also include, but does not always include, the understanding that these cells are immortal.

Murine pluripotent cells can be isolated from the preimplantation embryo and maintained in vitro as ES cells. ES cells retain pluripotence indefinitely and display the properties of stem cells, including competency to differentiate into all cell types, and the ability for indefinite self-renewal. Early primitive ectoderm-like (EPL) cells are also pluripotent stem cells. They differ in some properties to ES cells, and have the capacity to revert to ES cells in vitro. They can be derived from ES cells or other types of pluripotent cells, and are the in vitro equivalent of primitive ectoderm cells of postimplantation embryos. As such, EPL cells can also be established in vitro from cells isolated from the primitive ectoderm of postimplantation embryos. The properties of EPL cells, factors required for their maintenance and proliferation in vitro, and their ability to differentiate uniformly in vitro to form essentially homogenous populations of partially differentiated and differentiated cells types are described fully in PCT/AU99/00265, to applicants, the entire disclosure of which is incorporated herein by reference. Cells of the primordial gonad, primordial germ cells (PGCs), also retain pluripotency during embryonic development, and can be isolated and cultured in vitro as embryonic gonadal (EG) cells. Embryonic carcinoma (EC) cells may also be pluripotent.

The successful isolation, long term clonal maintenance, genetic manipulation and germ-line transmission of pluripotent cells from species other than rodents has generally been difficult to date and the reasons for this are unknown. International patent application WO97/32033 and U.S. Pat. No. 5,453,357 describe pluripotent cells including cells from species other than rodents, and primate pluripotent cells have been described in International patent applications WO98/43679 and WO96/23362 and in U.S. Pat. No. 5,843,780.

The differentiation of mouse ES cells can be regulated in vitro by the cytokine leukaemia inhibitory factor (LIF), which promotes self-renewal and prevents differentiation of the stem cells. In contrast to murine ES cells, human ES cells are not responsive to LIF. Therefore the maintenance of undifferentiated human ES cells to date has depended on the growth of the human cells on mouse “feeder” cells The requirement for feeder cells has placed limits the reproducibility and scalability of human ES cell production. Furthermore, there is some concern that the mouse feeder cells could provide a potential source of contamination of human ES cells with animal viruses, potentially making the ES cells unsuitable for therapeutic applications.

It is an object of the present invention to overcome, or at least alleviate, one or more of the difficulties or deficiencies associated with the prior art.

Accordingly, in an aspect of the present invention there is provided a method of regulating the differentiation potential of a pluripotent or pluripotent-related cell, which method includes

manipulating the expression and/or activity of a cell differentiation regulatory molecule in a pluripotent or pluripotent-related cell.

The cell differentiation regulatory molecule may be selected from one or more of the group consisting of a downstream biochemical target of LIF, an oncogene or oncoprotein, an upstream regulator of an oncogene or oncoprotein, and a molecule displaying similar activities.

Applicants have surprisingly discovered that the requirement for LIF in the regulation of differentiation of mouse pluripotent cells may be alleviated by the modification of the activity of regulatory molecules downstream of LIF signal transduction.

The method of regulation described above may be applied to facilitate maintenance and/or promote proliferation of pluripotent or pluripotent-related cells in vitro.

In a preferred embodiment of the present invention the expression and/or activity of the cell differentiation regulatory molecule is increased.

The pluripotent cells may be of any suitable type. Preferably, the pluripotent cells are selected from one or more of the group consisting of epiblast cells, ES cells, early primitive ectoderm-like (EPL) cells, primordial germ cells (PGCs), embryonic carcinoma (EC) cells and other pluripotent cells of embryonic origin.

The pluripotent or pluripotent-related cells may be murine of other mammalian, including human, origin.

In a further embodiment of the present invention, the pluripotent-related cells may include multipotent cells (such as haemopoietic stem cells and neural stem cells).

In a preferred embodiment, the cell differentiation regulatory molecule may be selected from one or more of the group consisting of the signal transducer and activator of transcription 3 (STAT3), a downstream biochemical target of STAT3 and a molecule displaying similar activities.

Accordingly, in this embodiment, there is provided a method of regulating the differentiation potential of a pluripotent or pluripotent-related cell, which method includes manipulating the expression and/or activity of a cell differentiation regulatory molecule selected from the group consisting of STAT3, a downstream biochemical target of STAT3 and a molecule displaying similar activities in a pluripotent or pluripotent-related cell.

Preferably the expression and/or activity of STAT3, the downstream biochemical target of STAT3 and/or the molecule displaying similar activities is increased.

In another preferred embodiment, the cell differentiation regulatory molecule is selected from one or more of the group consisting of a member of the myc family, an upstream regulator of a member of the myc family and a molecule displaying similar activities.

Accordingly, in this embodiment, there is provided a method of regulating the differentiation potential of a pluripotent or pluripotent-related cell, which method includes manipulating the expression and/or activity of a cell differentiation regulatory molecule selected from the group consisting of a member of the myc family, an upstream regulator of a member of the myc family and a molecule displaying similar activities in a pluripotent or pluripotent-related cell.

Preferably the expression and/or activity of the member of the myc family, upstream regulator of a member of the myc family, and/or molecule displaying similar activities is increased.

The expression and/or activity of the cell differentiation regulatory molecules may be manipulated by any suitable technique. Such techniques include but are not limited to manipulation of expression of the proteins, including manipulation of gene expression for example by transformation with expression constructs, antisense technology or fusion protein technology where the fusion protein includes a transduction domain linked to the cell differentiation regulatory molecule, or any other protein delivery system such as electroporation or lipofection. Such techniques are well known to those skilled in the art and are described in, for example, Sambrook et al (1989), the entire disclosure of which is incorporated herein by reference. A technique for the manipulation of the expression and/or activity of the cell differentiation regulatory molecule may also include the manipulation by use of a small molecule.

In a still further preferred embodiment of the present invention there is provided a method for the maintenance and proliferation of human pluripotent or pluripotent-related cells, which method includes manipulating the expression and/or activity of a cell differentiation regulatory molecule selected from one or more of the group consisting of a downstream biochemical target of LIF, an oncogene or oncoprotein, an upstream regulator of an oncogene or oncoprotein, and a molecule displaying similar activities, in a human pluripotent or pluripotent-related cell.

The method, in a preferred aspect, may be conducted in a medium in which the requirement for feeder cells to be included is substantially reduced or eliminated In another preferred embodiment the cell differentiation regulatory molecule may be selected from one or more of the group consisting of the signal transducer and activator of transcription 3 (STAT3), a downstream biochemical target of STAT3 and a molecule displaying similar activities.

Accordingly, in this embodiment, there is provided a method for the maintenance and proliferation of human pluripotent or pluripotent-related cells, which method includes manipulating the expression and/or activity of a cell differentiation regulatory molecule selected from one or more of the group consisting of STAT3, a downstream biochemical target of STAT3 and a molecule displaying similar activities, in a human pluripotent or pluripotent-related cell.

In yet another preferred embodiment the cell differentiation regulatory molecule may be selected from one or more of the group consisting of a member of the myc family, an upstream regulator of a member of the myc family and a molecule displaying similar activities.

Accordingly, in this embodiment, there is provided a method for the maintenance and proliferation of human pluripotent or pluripotent-related cells, which method includes manipulating the expression and/or activity of a cell differentiation regulatory molecule selected from one or more of the group consisting of a member of the myc family, an upstream regulator of a member of the myc family and a molecule displaying similar activities, in a human pluripotent or pluripotent-related cell.

In a further aspect of the present invention, there is provided a pluripotent or pluripotent-related cell produced by the methods according to the present invention.

The pluripotent or pluripotent-related cell may be of murine or other mammalian, including human, origin.

The pluripotent and pluripotent-related cells produced according to the present invention may have wide ranging applications.

For example they may have applications in therapies such as cell therapy, gene therapy, cancer therapy, regeneration and/or development of organs or appendages or limbs, production and/or use in pharmaceuticals and/or diagnostics and xenotransplantation.

In a further aspect of this invention, there is provided an embryo derived from a pluripotent or pluripotent-related cell produced by a method according to the present invention.

In a still further aspect of the present invention, there is provided a chimaeric or transgenic animal, including an animal derived by nuclear transfer, derived from an embryo formed from a cell produced by a method according to the present invention.

The present invention will now be more fully described with reference to the accompanying examples. It should be understood, however, that the description following is illustrative only and should not be taken in anyway as a restriction on the generality of the invention described above.

### EXAMPLE 1

### Maintenance and Proliferation of Pluripotent Cells in vitro by Manipulation of C-Myc: Gene Expression

Materials and Methods

c-myc Expression Construct:

A construct suitable for the stable expression of human c-myc was constructed by inserting a Eco RI fragment, from the plasmid pBpuroMycER (Littlewood et al. 1995) that contains the entire c-myc coding sequence fused to a modified estrogen receptor ligand binding domain, into the Eco RI site of the expression vector, pCAG-IRES-puro. This construct, pc-MycER.puro, utilizes the HCMVIEE fused to the chicken β-actin promoter and drives the transcription of the c-mycER open reading frame in addition to the puroR gene by way of an internal ribosome entry site (IRES).

Establishment of Stable c-myc Expressing Cells Lines:

20 μg of Pvu I-digested pc-MycER.puro was electroporated into 1×10⁷ D3 ES cells under the following conditions. Sub-confluent ES cells were trypsinized, washed once in PIBS and resuspended in 900 μl PIBS (1.1×10⁷ cells/ml) with DNA. Cells/DNA were placed in a Bio Rad electroporation cuvette and electroporated at 500 μF, 0.2 KV. Cells were immediately resuspended in 10 ml complete KO-DMEM in the absence of puromycin (Sigma). 2-3×10⁶ cells were then seeded into gelatinized 10 cm diameter tissue culture grade petri dishes in 10 ml fresh KO-DMEM plus KO-SR plus LIF in the absence of puromycin. After 18 hours, media was replaced with fresh complete KO-DMEM plus KO-SR plus LIF and 1 μg/ml puromycin. Cells were selected for over a 10-14 day period. Media was changed during selection every 24 hours. Clonal cell lines were initially amplified in gelatinized 24 well plates (Falcon) and expression of human c-mycER confirmed by Western blot analysis using the anti c-myc polyclonal antibody, N-262 (Santa Cruz) on crude whole cell lysates. Activation of c-MycER was accomplished be the addition of 4-Hydroxytamoxifen (Sigma) at a concentration of 100 nM to the KO-DMEM plus KO-SR media.

Preparation of Whole Cell Protein Extracts:

Whole cell extracts were prepared by washing cell pellets in ice cold PBS and resuspending in ice cold lysis buffer (250 mM NaCl, 50 mM Hepes pH 7.9, 0.1 mM EDTA, 0.1% NP-40, 1 mM DTT) at 1.5×10⁸ cells/ml. Protein concentrations of extracts were typically 2-4 mg/ml.

LIF Titration Assay:

LIF titration assays were set up in gelatinized 24 well trays. 900 μl of DMEM LIF was added to each well together with varying amounts of recombinant LIF to give final concentrations of 40-0 U/ml (ESGro, AMRAD). 500 cells were added to each well and the medium mixed for even spreading. After 6 days the plates were stained for alkaline phosphatase activity.

Alkaline Phosphatase Staining:

Alkaline phosphatase was visualised using the diagnostic kit 86-R (Sigma). The kit was used according to the manufacturer's specifications with the following modification; cell layers were fixed in 4.5 mM citric acid, 2.25 mM sodium citrate, 3 mM sodium chloride, 65% methanol and 4% para-formaldehyde prior to washing and staining.

Results

c-myc Drastically Reduces Spontaneous Differentiation of ES Cells

Examination of c-mycER expressing colonies in comparison to control (vector alone, puromycin-selected colonies or non-induced) revealed striking differences. First, stably-transfected c-mycER ES cell lines behaved indistinguishably from untransfected ES cells in the absence of 4-hydroxytamoxifen. Following addition of 4-hydroxytamoxifen they were far more rounded in shape. Besides forming spherical, domed-shaped colonies, a noticeable reduction in the number of differentiated cells surrounding the colonies was observed. This is in marked contrast to control ES cells which are prone to spontaneous differentiation (5-10% colonies on a plate normally exhibit a differentiated phenotype) in addition to the presence of differentiated cells at the periphery of colonies. Less than 1% of c-mycER induced cells exhibited a differentiated phenotype (data not shown). These characteristics were consistent in all ES colonies that expressed the, c-mycER transgene (4 c-mycER and 2 control cell lines were characterised).

c-myc Reduces the Requirement that IES Cells have for LIF

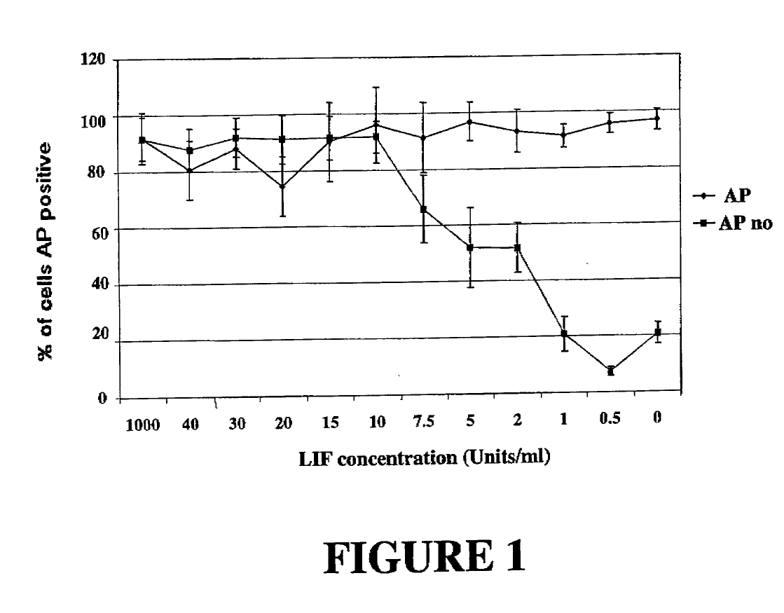

To evaluate the stability of c-mycER ES colonies and their ability to differentiate, we compared the ability of these cells to retain pluripotency in the presence of reduced levels of LIF. The logic being that if c-mycER was promoting pluripotency and blocking spontaneous differentiation, it may substitute, partially or fully, for LIF's stem cell maintenance function. This possibility was tested by growing control and c-mycER-transfected, puromycin-selected ES cells in the presence of LIF over a 40-0U/ml concentration range for 6 days. The assay was morphology-based and on the ability of ES colonies to retain alkaline phosphatase activity, a marker for pluripotency. The % of colonies scoring positive for alkaline phosphatase activity is represented in FIG. 1 in comparison to uninduced, puromycin-selected ES colonies. Clearly, c-mycER expressing colonies have a significant reduction in their requirement for LIF. Significant decreases in c-mycER ES cell pluripotency (as judged by alkaline phosphatase staining), were not seen until the LIF concentrations of 5U/ml and below (>75% alkaline phosphatase positive). Control ES cells generally lost pluripotency over the time course of this experiment at and below 20U/ml. These data clearly show that c-mycER expressing ES colonies have a significantly reduced requirement for LIF indicating that c-mycER has some stem cell stabilising function. The experiment shown is typical for more than 3 c-mycER lines tested in this assay. c-mycER expression levels in each pc-MycER.puro-transfected cell line were comparable (data not shown) as were the behaviour of the c-mycER cell lines by morphological and AP staining criteria.

Differentiation of Pluripotent Cells is Inhibited by c-myc

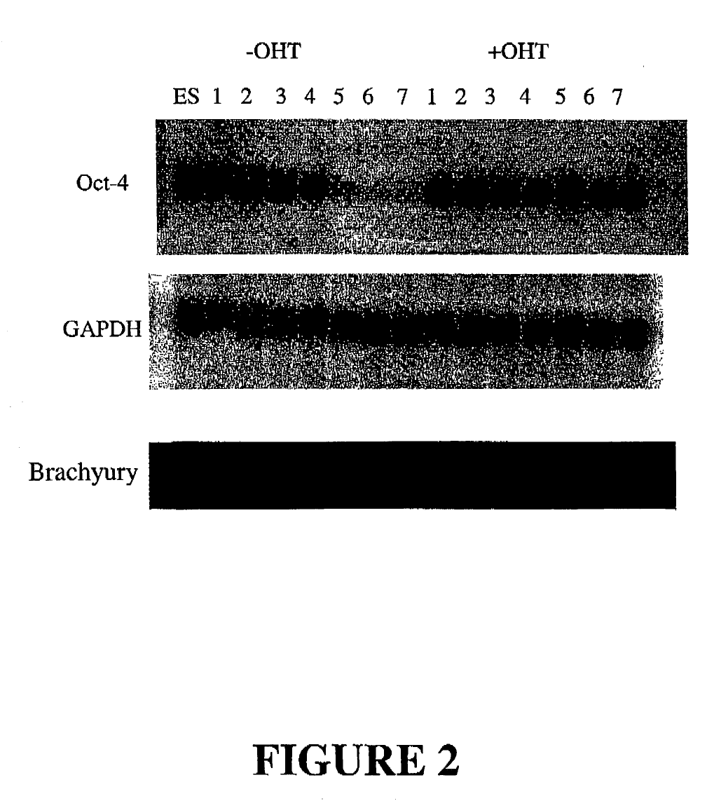

The ability of c-mycER to maintain ES cell pluripotency was characterised in further detail using conditions of cell growth that would normally facilitate the differentiation of cells, resulting in loss of pluripotency. The differentiation of uninduced versus induced c-mycER transfected cells was assessed by Northern blot analysis over a 7-day time course, using Oct4 (marker of pluripotency) and brachyury (marker for nascent mesoderm) probes. FIG. 2 shows that uninduced controls down-regulate Oct4 mRNA and lose pluripotency after 5 days (after LIF withdrawal) whereas all induced c-mycER cell lines tested maintain high levels of Oct4 expression throughout the experiment, confirming that they retain pluripotency. In control cell lines, brachyury mRNA was markedly up-regulated at days 4 and 5, indicating the formation of nascent mesoderm. In c-mycER cell lines, the expression of brachyury was typically not observed. This indicates that differentiation of c-myc expressing cells was incomplete and largely blocked. Hence, in the absence of LIF, the differentiation of pluripotent cells is severely compromised by the activity of c-mycER. This shows that c-mycER has properties that allow for the stabilisation maintenance of pluripotent stem cells.

It will be understood that the invention disclosed and defined in the specification extends to all alternative combinations of two or more of the individual features mentioned or evident from the text or figures. All of these different combinations constitute various alternative aspects of the invention.

It will be understood that the term “comprises” or its grammatical variants as used herein is equivalent to the term “includes” and is not to be taken as excluding the presence of other elements or features.

### REFERENCE

Littlewood T D, Hancock D C, Danielian P S, Parker M G, Evam G I. (1995) A modified oestrogen receptor ligand-binding domain as an improved switch for the regulation of heterologous proteins. Nucleic Acids Res. 23(10); 1686-90.

1. A method of regulating the differentiation potential of a pluripotent or pluripotent-related cell which method includes manipulating the expression and/or activity of a cell differentiation regulatory molecule in a pluripotent or pluripotent-related cell.

2. A method according to claim 1 wherein the cell differentiation regulatory molecule is selected from one or more of the group consisting of a downstream biochemical target of LIF, an oncogene or oncoprotein, an upstream regulator of an oncogene or oncoprotein, and a molecule displaying similar activities.

3. A method according to claim 1 wherein the method is applied to facilitate maintenance and/or promote proliferation of pluripotent or pluripotent-related cell in vitro.

4. A method according to claim 3 wherein the expression and/or activity of the cell differentiation regulatory molecule is increased.

5. A method according to claim 1 wherein said pluripotent or pluripotent-related cell is a cell of embryonic origin.

6. A method according to claim 5 wherein the pluripotent cell is selected from one or more of the group consisting of epiblast cells, ES cells, early primitive ectoderm-like (EPL) cells, primordial germ cells (PGCs), embryonic carcinoma (EC) cells and other pluripotent cells of embryonic origin.

7. A method according to claim 6 wherein the cell is selected from embryonic stem (ES) cells and early primitive ectoderm-like (EPL) cells.

8. A method according to claim 5 wherein said pluripotent and/or pluripotent-related cell is of mammalian origin.

9. A method according to claim 8 wherein said cell is of murine origin.

10. A method according to claim 8 wherein said cell is of human origin.

11. A method according to claim 1 wherein said pluripotent-related cell is a multipotent cell.

12. A method according to claim 11 wherein said multipotent cell is selected from haemopoietic stem cells and neural stem cells.

13. A method according to claim 1 wherein the cell differentiation regulatory molecule is selected from one or more of the group consisting of the signal transducer and activator of transcription 3 (STAT3), a downstream biochemical target of STAT3 and a molecule displaying similar activities.

14. A method of regulating the differentiation potential of a pluripotent or pluripotent-related cell, which method includes manipulating the expression and/or activity of a cell differentiation regulatory molecule selected from the group consisting of STAT3, a downstream biochemical target of STAT3 and a molecule displaying similar activities in a pluripotent or pluripotent-related cell.

15. A method according to claim 14 wherein the expression and/or activity of STAT3, the downstream biochemical target of STAT3 and/or the molecule displaying similar activities is increased.

16. A method according to claim 1 wherein the cell differentiation regulatory molecule is selected from one or more of the group consisting of a member of the myc family, an upstream regulator of a member of the myc family and a molecule displaying similar activities.

17. A method of regulating the differentiation potential of a pluripotent or pluripotent-related cell, which method includes manipulating the expression and/or activity of a cell differentiation regulatory molecule selected from the group consisting of a member of the myc family, an upstream regulator of a member of the myc family and a molecule displaying similar activities in a pluripotent or pluripotent-related cell.

18. A method according to claim 17 wherein the expression and/or activity of the member of the myc family, upstream regulator of a member of the myc family, and/or molecule displaying similar activities is increased.

19. A method for the maintenance and the proliferation of human pluripotent or pluripotent-related cells, which method includes manipulating the expression and/or activity of a cell differentiation regulatory molecule selected from one or more of the group consisting of a downstream biochemical target of LIF, an oncogene or oncoprotein, an upstream regulator of an oncogene or oncoprotein, and a molecule displaying similar activities, in a human pluripotent or pluripotent-related cell.

20. A method according to claim 19 wherein the method is conducted in a medium in which the requirement for feeder cells to be included is substantially reduced or eliminated.

21. A method for the maintenance and proliferation of human pluripotent or pluripotent-related cells, which method includes manipulating the expression and/or activity of a cell differentiation regulatory molecule selected from one or more of the group consisting of STAT3, a downstream biochemical target of STAT3 and a molecule displaying similar activities, in a human pluripotent or pluripotent-related cells.

22. A method for the maintenance and proliferation of human pluripotent or pluripotent-related cells, which method includes manipulating the expression and/or activity of a cell differentiation regulatory molecule selected from one or more of the group consisting of a member of the myc family, an upstream regulator of a member of the myc family and a molecule displaying similar activities, in a human pluripotent or pluripotent-related cell.

23. A pluripotent or pluripotent-related cell whenever produced by a method according to claim 1.

24. A pluripotent or pluripotent-related cell according to claim 25 which is of murine or other mammalian, including human, origin.

25. An embryo derived from a pluripotent or pluripotent-related cell produced by a method according to claim 1.

26. A chimaeric or transgenic animal, including an animal derived by nuclear transfer, derived from an embryo formed from a cell whenever produced by a method according to claim 1.

27. Use of a pluripotent or pluripotent-related cell according to claim 23 in therapies selected from any one of the group consisting of cell therapy, gene therapy, cancer therapy, regeneration and/or development of organs or appendages or limbs, production and/or use in pharmaceuticals and/or diagnostics, xeno transplantation, genetic modification of animals and nuclear transfer.
